(19) United States
(12) Patent Application Publication
(10) Pub. No.: US 2024/0226599 A1
(43) Pub. Date: Jul. 11, 2024

(54) PHOTOBIOSTIMULATION DEVICE FOR CELL REGENERATION

(71) Applicant: KARNAK MEDICAL S.r.L., MILANO (MI) (IT)

(72) Inventor: Elio MUTI, BERGAMO (BG) (IT)

(21) Appl. No.: 18/579,815

(22) PCT Filed: Jul. 6, 2022
(86) PCT No.: PCT/IB2022/056233

§ 371 Date: Jan. 16, 2024

(30) Foreign Application Priority Data
Jul. 16, 2021 (IT) 102021000018836

(51) Int. Cl. A61N 5/06; A61N 5/067

(52) U.S. Cl. CPC A61N 5/0613; A61N 5/067; A61N 2005/0627; A61N 2005/0638; A61N 2005/0642; A61N 2005/0652; A61N 2005/0659; A61N 2005/0663

(57) ABSTRACT

It is provided a photobiomodulation device (1) for cell regeneration of a subject (10) and comprising a support (2) including at least one emitter (3) configured to emit a light beam having a wavelength between 630 nm and 850 nm and defining an emission dose given by the product between light beam luminous intensity and light beam emission time, a control unit (4) operatively connected to the emitter (3), configured to allow a user at least to set the emission time and a predetermined dose, and configured to modulate the light intensity of the light beam in relation to the set emission time so that the emission dose corresponds to the predetermined dose, and acquisition means (5) operatively connected to the control unit (4) and configured to determine a distance between the emitter (3) and the subject (10) invested by the light beam, wherein the control unit (4) is further configured to modulate the light intensity of the light beam in relation to the acquired distance such that the emission dose corresponds to the predetermined dose.

The present invention relates to a photobiomodulation device for cell regeneration of the type specified in the preamble to the first claim.

In particular, the present invention has as its object a device capable of stimulating internal tissues, such as organs, bones, cartilage or others, or external tissues, such as the skin, or more generally parts of the body, such as limbs, head, torso or others, by means of red and/or near-infrared light emissions.

As is well known, the chlorophyll is a substance capable of collecting light thanks to its photosensitivity.

This property, believed until the end of the last century to be uniquely included in the plant kingdom, has always led doctors to recommend taking vegetables in the diet in order to make the most of exposure to sunlight or supplementing the diet by taking chlorophyll in capsules. Even earlier, it was noted that people suffering from osteo-articular and muscular pains, as well as rheumatic problems, improved their condition by exposing themselves to the sun, a practice also known as heliotherapy.

Only in more recent years and, in particular, at the beginning of the new millennium have even more in-depth studies been conducted on the interaction of light with human and animal cells. In particular, it was discovered that even in human and animal cells there is a photosensitive substance capable of functioning in cells like chlorophyll does for plants: Cytochrome-c oxidase, also known by the acronym COX/CCO.

This substance is located within the mitochondria of every cell. The mitochondrion, as is well known, is nothing more than the power station of the cell and in terms of both structure and function is entirely analogous to the chloroplast of plants.

The function of the mitochondrion is to create energy in the form of ATP, or Adenosine Triphosphate, so that the cell can fully perform its intended function and work efficiently. Therefore, the more energy it produces, the higher the value of the electrical voltage differential between the inside of the cell and the outside.

This value, known as the cell membrane potential, is the parameter that allows us to measure, on a biophysical level, the degree of cell viability.

For example, it is considered that the cell basically operates with full vitality between values of −70 and −90 mV and that, approaching zero, the cell progressively loses functionality until it becomes diseased around the value of −40 mV and can even degenerate at −20 mV.

Therefore, it is evident that energy is an essential requirement for life.

The cells are only able to capture the energy of light when this source is able to enter tissue. The humans and animals, unlike plants, do not have leaves to point in the direction of light in order to capture the right amount of energy, yet their organisms are equipped with an organ that is more diffuse in the body, namely the skin.

Much of the sunlight is unable, however, to enter the inner layers of the skin, since the human and animal envelope is composed of several layers, namely the epidermis, dermis, subcutaneous tissues, muscles, bones and inner tissues.

Generally, most sunlight stops at the outermost part and sunburn occurs precisely for this reason. The inability to penetrate the tissues means, therefore, that all the energy potential falls on the outermost part which, stressed, becomes damaged. This is, in particular, the case with ultraviolet rays.

There are, however, gradations of light that are able to pass through the skin and also reach the organs. The different gradations, as is known, are scientifically dependent on the ‘wavelength’ parameter and refer to both visible light radiation and radiation invisible to the human eye.

The most interesting wavelengths for the health of cells and the body are, in detail, those relating to red and near infrared, or NIR.

These wavelengths are scientifically recognised for their interaction with the cells of living beings and are, in fact, used for low-intensity light therapy: Low Level Light Therapy, or LLLT, also known as photobiostimulation or photo-biomodulation.

A technology capable of generating these wavelengths makes it possible to activate cellular energy production via the mitochondria and COX/CCO so that, in the right doses, this interaction is able to increase not only ATP energy production but also the number and size of mitochondria.

Naturally, in addition to the fact that different cell windows open at different wavelengths, dosage is extremely important in order to achieve an activating, rather than inhibiting, effect on, for example, cellular ATP energy production.

The devices known in the current state of the art that are capable of performing photobiomodulation of a body generally allow the intensity, rather than the wavelength, of the emission to be altered without taking into account other parameters that may affect not only the effectiveness of the dose of light administered, but also the health of the user himself.

In addition, a further drawback of the known technique is that the modulation of light is generally static and does not follow, for example, the movements to which the user may be subjected or other features relevant to the dynamic administration of light doses.

In this situation, the technical task underlying the present invention is to devise a photobiomodulation device for cell regeneration capable of substantially obviating at least part of the aforementioned drawbacks.

In the context of said technical task, it is an important aim of the invention to obtain a photobiomodulation device for cell regeneration which takes into account all the parameters necessary for an effective and safe administration of the correct dose of light.

Another important aim of the invention is to achieve a photobiomodulation device for cell regeneration which also takes into account, during the administration of a dose of light, the movements to which the user is subjected.

In conclusion, a further task of the invention is to realise a photobiomodulation device for cell regeneration which is capable of dynamically modulating the light emission.

The specified technical task and purposes are achieved by a photobiomodulation device for cell regeneration as claimed in the annexed claim 1.

Preferred technical solutions are disclosed in the dependent claims.

The features and advantages of the invention are hereinafter clarified by the detailed description of preferred embodiments of the invention, with reference to the appended drawings, in which:

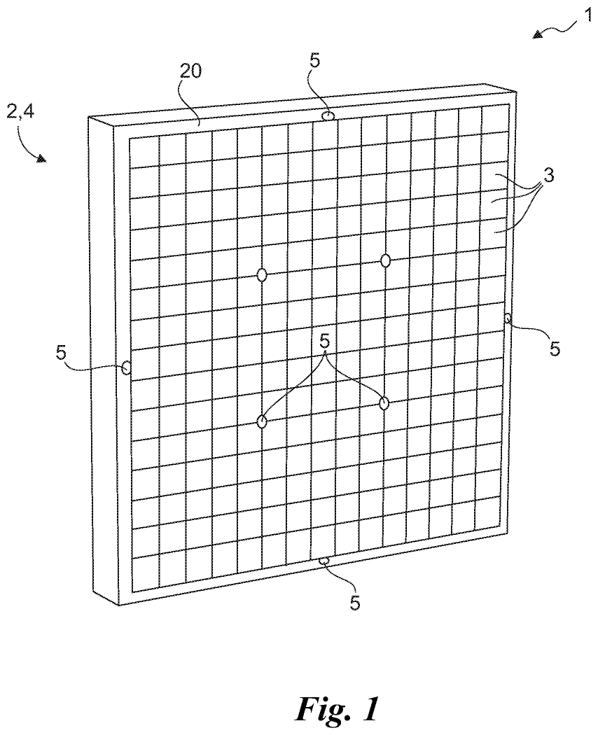

the FIG. 1 illustrates a perspective view of a first embodiment of a photobiomodulation device for cell regeneration according to the invention;

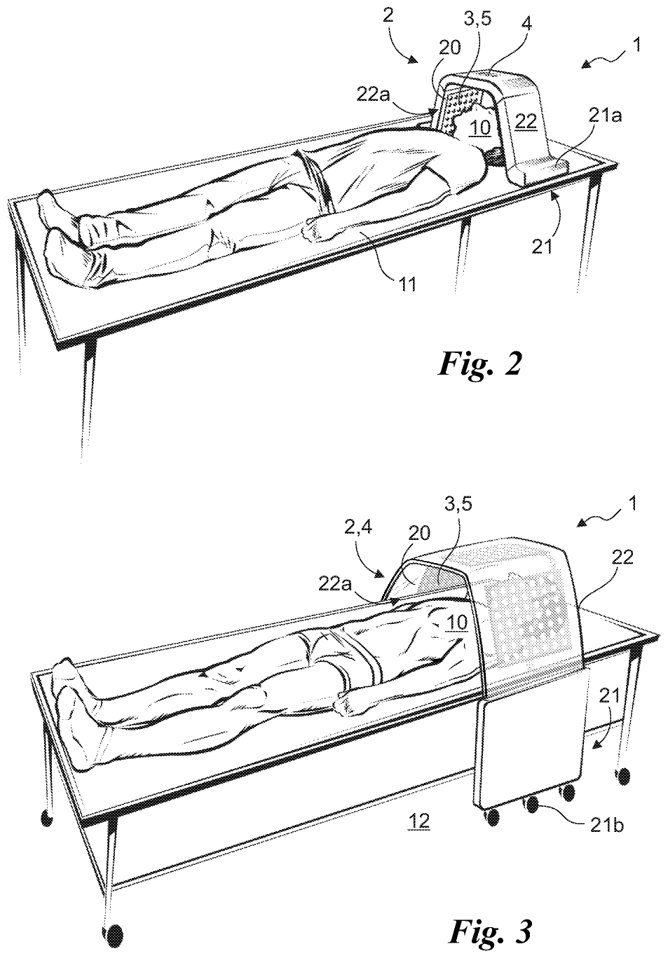

the FIG. 2 illustrates a perspective view of a second embodiment of a photobiomodulation device for cell regeneration according to the invention;

the FIG. 3 is a perspective view of a third embodiment of a photobiomodulation device for cell regeneration according to the invention; and

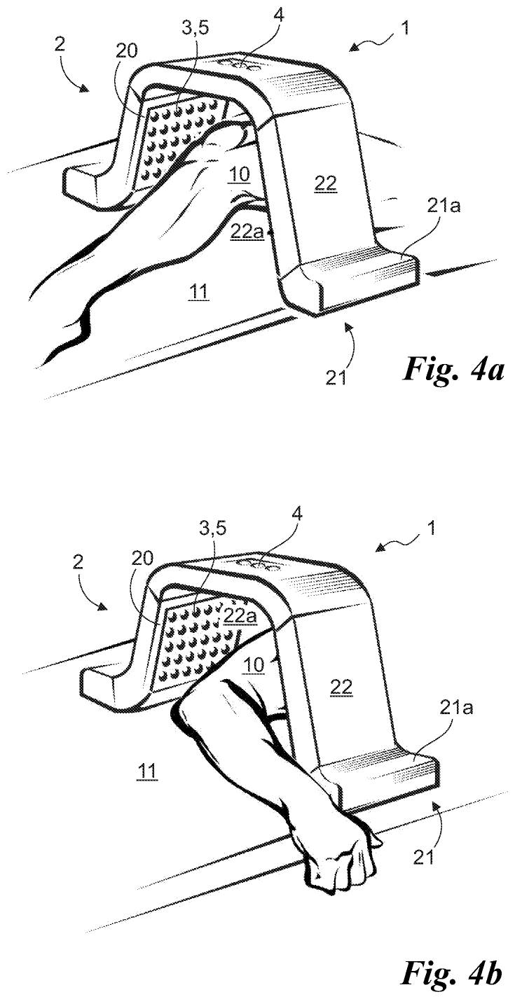

the FIG. 4a represents a first example of use of the device of FIG. 2 wherein a leg of a user is stimulated;

the FIG. 4a illustrates a second example of use of the device of FIG. 2 in which an arm of a user is stimulated; and

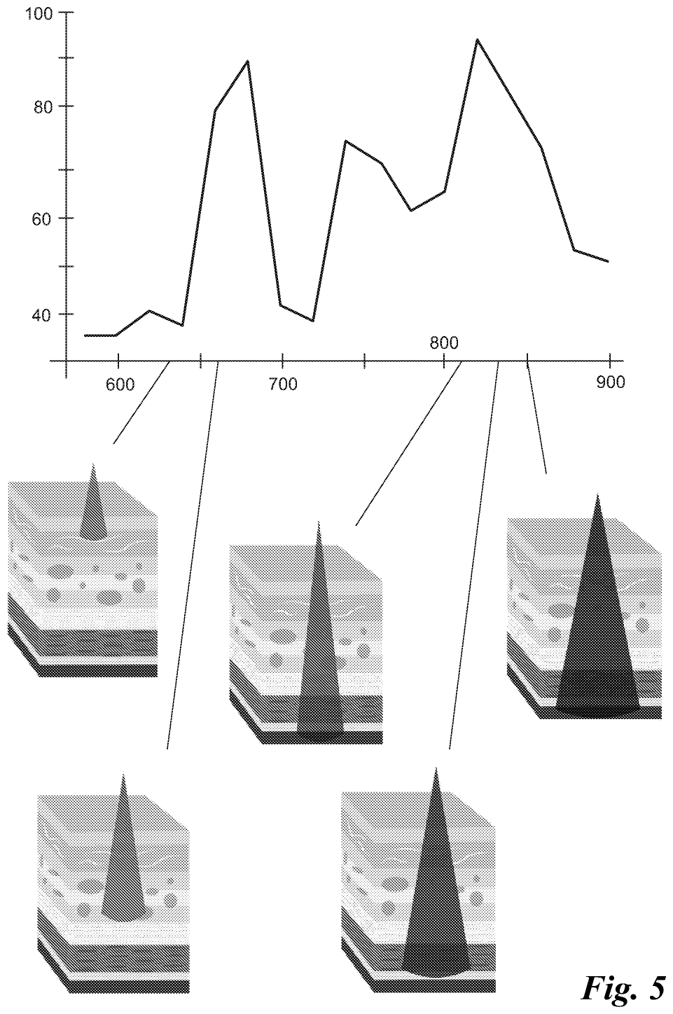

the FIG. 5 illustrates a wavelength graph of activation of cytochrome-c oxidase wherein the abscissa shows the wavelength in nm, the ordinate shows the percentage of activated cells, and in which examples of tissue penetration are shown depending on the wavelength of electromagnetic emission.

In the present document, the measurements, values, shapes and geometric references (such as perpendicularity and parallelism), when associated with words like “about” or other similar terms such as “approximately” or “substantially”, are to be considered as except for measurement errors or inaccuracies due to production and/or manufacturing errors, and, above all, except for a slight divergence from the value, measurements, shape, or geometric reference with which it is associated. For instance, these terms, if associated with a value, preferably indicate a divergence of not more than 10% of the value.

Moreover, when used, terms such as “first”, “second”, “higher”, “lower”, “main” and “secondary” do not necessarily identify an order, a priority of relationship or a relative position, but can simply be used to clearly distinguish between their different components.

Unless otherwise specified, as results in the following discussions, terms such as “treatment”, “computing”, “determination”, “calculation”, or similar, refer to the action and/or processes of a computer or similar electronic calculation device that manipulates and/or transforms data represented as physical, such as electronic quantities of registers of a computer system and/or memories in, other data similarly represented as physical quantities within computer systems, registers or other storage, transmission or information displaying devices.

The measurements and data reported in this text are to be considered, unless otherwise indicated, as performed in the International Standard Atmosphere ICAO (ISO 2533:1975).

With reference to the Figures, the photobiomodulation device for cell regeneration according to the invention is globally referred to as 1.

The device 1 is preferably used for therapy type applications, for example low intensity light therapy: Low Level Light Therapy, or LLLT, or for aesthetic medicine, wellness or simply home use.

The device 1 comprises, in essence, a support 2.

The support 2 is essentially a physical element capable of supporting most, or even all, of the components of the device 1.

In particular, the support 2 includes at least one emitter 3.

The emitter 3 is configured to emit a light beam i.e. a light radiation. Thus, for example, the emitter 3 may be of the type of at least a choice between LED, emitting non-coherent light, and Laser, emitting coherent light. Of course, emitter 3 could also include other types of light sources.

Even more preferably, the emitter 3 is preferably configured to emit a light beam having a wavelength between 630 nm and 850 nm.

Even more preferably, the emitter 3 may be configured to emit light beams having a wavelength between ranges of 630-660 nm and 810-850 nm, respectively.

It is important to note that light beams whose wavelength is between 630 nm and 660 nm are essentially known as Deep Red light, while light beams whose wavelength is between 810 nm and 850 nm are essentially known as NIR.

As shown in FIG. 5, the Deep Red light beams are particularly suitable for penetrating the superficial layer of the human body and treating the skin, while NIR light beams are suitable for penetrating beyond the skin layer and reaching internal body parts, e.g. organic tissues, cells, neurons and bones.

The emitter 3, of course, can be powered in any way, i.e. direct current or even alternating current. For example, the light beam emitted by emitter 3 can be generated with 100% duty cycle or even 50% duty cycle in square wave.

Furthermore, the light beam can be generated at certain frequencies, for example preferably, but not only, in the frequencies between 10 Hz and 100 Hz.

The emitter 3, by emitting a light beam with its own wavelength and, of course, its own intensity, also defines an emission dose.

The emission dose is essentially the amount of light administered over time to a subject 10 exposed to emitter 3.

Therefore, the emission dose is the product of the light intensity of the light beam and the emission time of the same light beam.

The term subject 10 is of course to be understood in a generic sense, i.e. that it does not necessarily refer to an inanimate object, but may also include a part of the human body or even the whole human body.

Thus, the subject 10 may be any inanimate element, any living being, e.g. human, animal or plant, and may also correspond to a part of a living being, as indeed shown in the FIGS. 4a-4b.

The support 2 therefore substantially supports at least the emitter 3.

Furthermore, the support 2 could also include a plurality of emitters 3.

Such emitters 3 could be neatly distributed in such a way as to make a grid or surface in which the emitters 3 are positioned so as to be mutually equi-spaced.

In general, the support 2 comprises at least one emission wall 20.

The emission wall 20 is a part of the support 2 including one or more emitters 3.

The emission wall 20 may therefore be flat, or even curved.

The emission wall 2 is preferably configured to face the subject 10.

Eventually, the emission wall 20 may be configured to wrap around the subject 10 itself.

The support 2 may thus be realised in a variety of ways.

In general, the support 2 is configured to be stably constrained, by interlocking or even just resting, at a plane. For example, the plane could be a wall, a ceiling, a floor or even a ground or something else.

Preferably, the support 2 is spaced apart, when in use, from the subject 10. Thus, the support 2 is configured to support one or more emitters 3 in such a way that they are spaced apart, i.e. not in contact, with respect to the subject 10.

In a first embodiment, the emission wall 20 may substantially comprise a panel on the surface of which emitters 3 are substantially arranged and ordered, as shown in FIG. 1.

Thus, the emission wall 20 may be easily positioned either on walls, ceilings and, in general, on any flat surface that may allow the emission wall 20, i.e. the support 2, to be supported.

Alternatively, the support 2 may comprise at least two support ends 21 and a frame 22.

The frame 22 is essentially a structural element, preferably arched, connecting the support ends 21.

Thus, the frame 22 preferably defines a housing 22a.

The housing 22a is substantially the space bounded, at least in part, by the frame 22 and part of the support ends 21.

Thus, the housing 22a is substantially the space subtended by the arched frame 22.

Thus, the frame 22 may comprise the emitter wall 20. The latter advantageously faces the housing 22a, or rather one or more emitters 3 are configured, supported by the frame 22, to emit light towards the housing 22a.

In a second embodiment of the device 1, each of the two support ends 21 may comprise a foot 21a.

Each foot 21a is substantially configured to rest the support 2 stably on a support surface 11.

The support surface 11 is substantially any surface on which a subject 10 may rest. Thus, the support surface 11 may, for example, be part of a cot, rather than a floor, a bench, or something else.

The foot 21a is substantially an element provided, for example, with a flat surface capable of interfacing, in support, with the support surface 11.

In a third embodiment, at least one of the support ends 21, possibly even each of them, may comprise handling means 21b.

The handling means 21b are preferably configured to allow movement of the support 2 relative to a sliding plane 12.

The sliding plane 12 is essentially any plane on which the support 2 may rest.

Thus, the sliding surface 12 may, for example, be part of a cot, rather than a floor, a bench, or even more.

Furthermore, the sliding surface 12 may also coincide with the support surface 11.

The sliding means 12, in particular, are configured to allow movement of the support 2 along at least one predetermined direction.

The handling means 12 may therefore include wheels oriented along the same direction.

Naturally, the handling means 21b could also be effectively combined with the first embodiment, and could for example be arranged between the emission wall 20 and the sliding surface 12 on which the wall 20 moves.

The device 1 preferably also comprises a control unit 4.

The control unit 4 is operatively connected to the emitter 3, or to the emitters 3 if the latter are a plurality.

Thus, the control unit 4 is configured to allow a user at least to set the emission time. This means that the control unit 4 allows the user to indicate to the device 1 how long it is required for the emitter 3 to strike the subject 10 with the light beam.

Furthermore, the control unit 4 is configured to also allow a predetermined dose to be set. The predetermined dose is preferably the maximum administration dose for the subject illuminated by the device 1.

Naturally, the dose may vary depending on the type of subject 10 facing the emission wall 20 and/or in the housing 22a.

The control unit 4, therefore, is configured to modulate the light intensity of the light beam in relation to the set emission time so that the emission dose corresponds to the predetermined dose,

This means that the control unit 4 is able to calculate, autonomously, by how much it is necessary to reduce or increase the light intensity in relation to the set emission time, knowing the predetermined dose to be administered to the subject 10.

As is evident, since the emission dose is given by the product of the light intensity of the light beam and the emission time of the light beam, for high times, the light intensity is reduced, while for low times, the light intensity is high.

The control unit 4, structurally, may be any computer capable of controlling the other components, such as the emitters 3, and capable of receiving and sending information. Furthermore, the control unit 4 is preferably capable of performing calculations on its own and may therefore include an electrical board or other equivalent elements.

The control unit 4 could further comprise an interface and/or display accessible from outside the holder 2 so as to facilitate control of the control unit 4 by the user.

For example, a push-button panel or a graphical display interface could be provided so as to realise a manipulatable touch screen.

In general, the control unit 4 may also be configured to allow the user to select said wavelength between ranges of 630-660 nm and 810-850 nm, or rather by activating Deep Red and/or NIR emission, respectively.

Alternatively, also, the control unit 4 may include connecting means.

If present, the connection means are preferably configured to allow remote connection of the control unit 4 with an external processor.

The external processor may, at the same time, be configured to allow the user to issue commands to the control unit 4 from external regarding to the support 2.

The external processor may thus be a smartphone, tablet, PC or other equivalent element capable of communicating with the control unit 4 by means of connection media. The latter, in detail, may be of the Wi-Fi, Bluetooth™, IR or other types.

Advantageously, device 1 comprises further elements.

Indeed, device 1 also comprises acquisition means 5.

The acquisition means 5 are operatively connected to the control unit 4. Thus, advantageously, the acquisition means 5 are configured to determine a distance between the emitter 3 and the subject 10 invested by the light beam.

The acquisition means 5 may thus be simple proximity sensors or other sensors capable of determining the distance of the subject 10 with respect to the emitter 3 or the emission wall 20.

For example, the acquisition means 5 may comprise a time-to-flight type sensor.

The acquisition means 5 may be positioned in the vicinity of the emitters 3, for example at the grid made by the emitters 3, or also around the emitters 3. In general, the acquisition means 5 may be positioned in any position on the emission wall 20 so as to face the subject 10, as well as the emitters 3.

Advantageously, the control unit 4 is further configured to modulate the light intensity of the light beam in relation to the distance acquired, via the acquisition means 5, so that the emission dose corresponds to the predetermined dose.

Thus, the device 1 is able to modulate the intensity not only in relation to the set emission time, but also in relation to the distance between the subject 10 and the emitter 3, i.e. the emission plane 20.

Even more advantageously, the acquisition means 5 can be configured to acquire the distance in time during at least the emission time. The control unit 4 can then be configured to modulate the light intensity in a time-varying manner in relation to changes in the acquired distance over time such that the emission dose corresponds to the predetermined dose.

In other words, the acquisition means 5 and the control unit 4 can be configured to modulate the intensity of the light beam in real time, i.e. dynamically throughout the duration of the emission time, so as to compensate for any variation in the distance between subject 10 and emitter 3 for example due to relative movement between subject 10 and support 2.

The operation of the device 1 described above in structural terms is as follows. Basically, after having set the predetermined dose and the emission time, it is possible to arrange the subject 10 in front of the emitter 3, for example in front of the emission wall 20, in the first embodiment, or in the housing 22a, in the other embodiments, in order to allow the photobiomodulation of the subject 10 itself. The parameters of emission time, emission type and predetermined dose are settable and, therefore, the emission dose can be fully controlled by the user. However, the modulation of the dose is performed by the device in real time, respecting the parameters set by the user, so that the desired bio-stimulation is performed in total efficiency and safety and without requiring any effort or expertise from the user.

The invention also enables a new procedure for setting up photobiomodulation for cell regeneration of a subject 10.

The process comprises, in brief, at least one setting step.

In the setting step, the emission time of the light beam having a wavelength between 630 nm and 850 nm is set, which defines an emission dose given by the product of the light intensity of the light beam and the emission time of the light beam.

In addition, a predetermined dose is set in the setting phase.

Thus, the procedure also includes a modulation step in which the light intensity of the light beam is modulated in relation to the set emission time so that the emission dose corresponds to the predetermined dose.

The procedure, advantageously, further comprises a determination phase. In the determination step, the distance of the emitter 3 and the subject 10 hit by the light beam is determined.

Then, advantageously, in the modulation step, the light intensity of the light beam is also modulated in relation to the acquired distance so that the emission dose corresponds to the predetermined dose.

The photobiomodulation device for cell regeneration 1 according to the invention achieves, therefore, important advantages.

Indeed, the device 1 takes into account, during operation, all the parameters necessary for the effective and safe administration of the correct dose of light.

Furthermore, the stimulation is also controlled dynamically, so that any damage or incorrect administration of light doses is prevented.

Thus, the device 1 is able to manage the administration in any situation and on any type of subject, as all parameters can be set by the user, possibly even remotely with an external computer.

For example, the device 1 lends itself to the most diverse uses, e.g. inside a gymnasium, an environment in which a subject can occupy a specific area for an appropriate amount of time to complete their exercises. The device 1 can be configured to illuminate the specific area, e.g., constrained to a wall or ceiling, and can be easily configured by the subject to receive the treatment during his or her workout, thus also while on the move, as the control unit is able to modulate the parameters according to what is pre-set and the distance, even dynamically.

The invention is susceptible to variations within the scope of the inventive concept as defined by the claims.

Within that scope, all details are substitutable by equivalent elements and the materials, shapes and dimensions can be any.

1. Photobiomodulation device for cell regeneration in a subject comprising:
a support including at least one emitter configured to emit a light beam having a wavelength between 630 nm and 850 nm and defining an emission dose given by the product between the luminous intensity of said light beam and the emission time of said light beam,
a control unit operatively connected to said emitter, configured to allow a user at least to set said emission time and a predetermined dose and configured to modulate said luminous intensity of said light beam in relation to said emission time set in such a way that said emission dose corresponds to said predetermined dose,
and further comprising
acquisition means operatively connected to said control unit and configured to determine a distance between said emitter and said subject hit by said light beam,
said control unit being further configured to modulate said light intensity of said light beam in relation to said acquired distance so that said emission dose corresponds to said predetermined dose.

2. Device according to claim 1, wherein said acquisition means acquire said distance in time during at least said emission time and said control unit is configured to modulate said light intensity in a variable manner in time in relation to variations of said distance acquired over time in such a way that said emission dose corresponds to said predetermined dose.

3. Device according to claim 1, wherein said emitter is of type choice between LED and Laser.

4. Device according to claim 1, wherein the control unit is configured to allow a user to select said wavelength between intervals respectively between 630-660 nm and 810-850 nm.

5. Device according to claim 1, wherein said support comprises a plurality of said emitters.

6. Device according to claim 1, wherein said support comprises at least one emission wall including one or more of said emitters and configured to appear on said subject.

7. Device according to claim 1, wherein said support comprises at least two support ends and a frame to arch connecting said support end defining a housing adapted to house said subject and partly delimited by said frame and part of said support ends and comprising said emission wall facing towards said housing.

8. Device according to claim 1, wherein each of said support end comprises a foot configured for resting said support stably on a support surface for said subject.

9. Device according to claim 1, wherein at least one of said support end comprises movement means configured to allow the movement of said support with respect to a sliding plane along at least one predetermined direction.

10. Procedure for setting a photobiomodulation for cell regeneration of a subject comprising:
setting an emission time of a light beam having a wavelength between 630 nm and 850 nm and defining a given emission dose from the product between the luminous intensity of said light beam and the emission time of said light beam and a predetermined dose,
modulate said light intensity of said light beam in relation to said emission time set in such a way that said emission dose corresponds to said predetermined dose,
and further comprising
determining a distance between said emitter and said subject hit by said light beam, and
modulating said light intensity of said light beam in relation to said acquired distance such that said emission dose corresponds to said predetermined dose.
